(19) United States
(12) Patent Application Publication
(10) Pub. No.: US 2019/0200553 A1
(43) Pub. Date: Jul. 4, 2019

(54) Method for Producing Rice Haploid by Rice X Maize Hybridization

(71) Applicant: Institute of food crops, Yunnan Academy of Agricultural Sciences, Kunming City (CN)

(72) Inventors: Mingliang Ding, Kunming City (CN); Mujun Yang, Kunming City (CN); Hongsheng Li, Kunming City (CN); Shaoxiang Li, Kunming City (CN); Kun Liu, Kunming City (CN); Jian Gu, Kunming City (CN); Hong Zhao, Kunming City (CN); Zhonghui Yang, Kunming City (CN)

(21) Appl. No.: 16/196,842

(22) Filed: Nov. 20, 2018

(30) Foreign Application Priority Data
Dec. 28, 2017 (CN) 201711461575.3

(51) Int. Cl. A01H 1/08; A01H 6/46

(52) U.S. Cl. CPC A01H 1/08; A01H 1/02; A01H 6/4684; A01H 6/4636

(57) ABSTRACT

The present invention provides a novel method for producing the rice haploid, i.e., producing the rice haploid by rice×maize hybridization. In this method, rice is used as the female parent, and the rice panicle is emasculated and then pollinated with fresh maize pollens; the emasculated panicle is sprayed with 2, 4-D solution at 50-200 mg/L 24 h after pollination, and after 15-20 days the rice panicle is cut off to collect caryopses; and the haploid embryos are obtained by dissectting caryopses asepticlly, then inoculated and cultured with ½MS medium, then the haploid embryos directly germinate into rice haploid seedlings. Compared with the existing main methods for producing the rice haploid, such as anther culture and isolated microspore culture, the rice haploid production method of the present invention reduces the dependence on rice genotypes, does not produce mixture of haploid and diploid plants, contains no albino seedling, and is simpler in technical operation.

### CROSS REFERENCE TO RELATED APPLICATION

This application claims the benefit of and takes priority from Chinese Patent Application No. 201711461575.3 filed on Dec. 28, 2017, the contents of which are herein incorporated by reference.

### TECHNICAL FIELD

The present invention belongs to the technical field of crop genetics and breeding, and in particular relates to a method for producing rice haploids through wide cross between rice and maize.

### BACKGROUND

Rice is a main food crop in the world, and is also the first majority food crop in China. The sustainable development of rice research and production is of great strategic significance for safeguarding the food security in the world and in China. Conventional breeding technologies have developed a large number of excellent rice varieties for agricultural production, greatly improved rice yield and quality, and made significant contributions to the improvement of people's lives and the development of national economy. However, for the conventional breeding method, the cycle is long, the workload is large, and it takes 6-8 years or even longer to breed a variety. Due to constant changes in market demand, pathogenic physiological races and production environment, a variety obtained for a long time period may no longer be able to meet the requirements of actual consumption and production, resulting in waste of time and resources. Therefore, accelerating the breeding process and shortening the breeding time are one of the key points for breakthroughs in rice breeding techniques and methods.

Doubled haploid (DH) technology in crops belongs to cell and chromosome engineering technologies, is a new breeding technology which is fast, efficient and safe, and mainly has 4 major outstanding advantages: being fast and stable, enabling any breeding progeny material be homozygous and stable in one generation; having a wide application range, where the DH breeding technology can be randomly combined with all other breeding methods; significantly reducing the dependence on breeding experience; and accelerating ground-breaking excellent germplasm innovation. The DH technology is one of the main high-efficiency breeding technologies.

A DH plant is derived from a haploid plant by chromosome doubling, and thus in order to obtain a rice DH, a haploid plant must first be obtained. At present, the methods for producing rice haploids are mainly anther culture and isolated microspore culture.

Anther in vitro culture is one of the main methods for producing rice haploids. In 1968, Niizeki et al. first obtained rice haploids by anther culture. About 30%-40% of haploids obtained by anther culture can be naturally doubled (Mishra et al., 2013). Rice anther culture mainly includes two main steps: callus induction and regeneration of embryogenic callus (also called embryoid body), generally, the process is as follows: during the booting stage of rice, in the mid to late periods of the maturing of microspores, anthers of the rice panicle are collected for cultivation; the leaf sheath is wiped with a clean cotton cloth dipped in 70% alcohol (the young spike was still in the closed leaf sheath); then the leaf sheath is pretreated at 10° C. for 8-10 days, and ready for use; the leaf sheath is peeled to take out the spike, and the surface of the spike is disinfected with 20% bleaching water (containing 4% sodium hypochlorite) for 5 min, and then rinsed with sterile deionized water for three times; then the microspores are subjected to microscopic examination, and 20-25 pollen grains at the middle-uninuclear stage of microspores are evenly spread on the surface of a culture medium; the inoculated anthers are darkly cultured at 25±1° C., and the condition of anther-induced callus generation is observed 3-4 weeks after the inoculation; then the callus is transferred into a regeneration medium under the condition of 25±1° C. and cultured under light (about 2000 lux) to induce the formation of regeneration seedlings; then the obtained green plantlets are transferred into a rooting medium for rooting culture; and finally plants with good root structures are transferred into flowerpots in a greenhouse until maturity.

The main disadvantages of existing methods for generating a rice haploid through anther culture are:

1) having high genotype dependence: that is, only a part of rice materials (in general conditions, different materials have different genotypes) can produce more haploid plants by this method; for other materials, the production frequency of haploid plants is extremely low, or alternatively no haploid plant can be produced at all; because the rice breeding involves thousands of materials of different genotypes, and it is entirely possible to occur that: materials with excellent performance are difficult to produce haploids, while materials with poor performance can produce haploids;
2) the obtained regeneration plants are not necessarily all haploids, and some of them may be diploid cells derived from pollen walls, tapetal layers, etc., and the application values of these plants are relatively lower;
3) even if the regenerated plants are obtained from haploid microspores rather than cells derived from the pollen walls or tapetal layers, the obtained regeneration plants are a mixture of haploids and doubled haploids due to the chromosome natural doubling of different frequencies during the process of producing green plantlets, rooting and sprouting, and thus it is difficult to distinguish the regeneration plants one by one before a chromosome doubling treatment of the regeneration plants, such that among the plants obtained after the chromosome doubling treatment, some may be doubled haploids, and some may be tetraploids, reducing the practical application effect of the whole method; and
4) other problems: such as anther browning (referring to the condition that anthers release brown materials during culture, and anthers are gradually browning and then died) and plant albino (i.e., albino seedlings, referring to the condition that seedlings obtained from isolated culture are subjected to whole-plant chlorosis or partial chlorosis), the seedlings are presented as white or yellow, and finally died due to the inability of performing photosynthesis.

Isolated microspore culture is another rice haploid production pathway established on the basis of anther culture. The main difference in operation method between the isolated microspore culture and the anther culture is that, a procedure of collecting isolated microspores is added in the former one, and that is, the anthers are broken by physical methods such as extrusion, grinding and the like to release isolated microspores, and then the isolated microspores are collected by operations such as filtration, centrifugation and the like (removing diploid cells such as cells derived from the pollen walls, the tapetal layers and the like), through these treatments it is ensured that the finally obtained regeneration plants are derived from haploid microspore cells, and then the microspores are inoculated onto a medium for cultivation; and other technical operations of the isolated microspore culture are similar to that of the anther culture. The latter one (anther culture) is directly inoculating anthers onto a medium for cultivation.

The shortcomings of the rice isolated microspore culture are basically similar to those of the anther culture, and are mainly presented as follows:

1) having high genotype dependence, and that is only a part of rice materials can produce haploid plants by this method;
2) since the chromosome natural doubling of different frequencies occur during cultivation of the regenerated haploid green plantlets, the obtained regeneration plants are a mixture of haploids and doubled haploids, where the haploid plants can only produce seeds after subjecting to the chromosome doubling treatment, while the doubled haploids can bear fruits without the chromosome doubling treatment, and however for the resultant thousands of mixed regeneration plants consisting of haploids and doubled haploids, the workload is extremely large to differ the haploid seedlings from such mixture before chromosome doubling treatment; if all regenerated plants are subjected to chromosome doubling treatment without the identifying, then among the obtained plants, some may be doubled haploids, and some may be tetraploids; and if the chromosome doubling treatment is not performed, then it is equivalent to discarding haploid seedlings (the haploid seedlings will be unable to bear seeds and breed offspring), Therefore, the application effect of the method in actual breeding is reduced;
3) a certain frequency of albino seedlings will also appear;
4) the procedure is more complicated than that of the anther culture, and accordingly increased the cost.

In view of the above, in anther culture or microspore culture, most rice materials have a poor effect in producing haploids, where the callus formation rate is low, the regenerated plants are of a small number and a poor quality, the albino seedlings are of relatively greater number, and the efficiency of producing haploids or doubled haploids is low, and thus the application of the anther culture or microspore culture in rice breeding is extremely limited. Therefore, creating new rice haploid production pathway is of great significance for improving rice breeding efficiency theoretically and practically.

### SUMMARY

In view of the main problems existed in current rice haploid production methods such as the anther culture and isolated microspore culture, the present invention provides a novel method for producing rice haploids, i.e., obtaining haploid embryos containing only the rice genome by the elimination of maize genome through hybridization between rice and maize, and then obtaining the rice haploid by embryo rescue. The method for producing the rice haploid by rice×maize hybridization includes the following steps:

A. emasculation: after heading and before flowering of the rice, for each panicle, cut off young base spikelets and keep upper and middle spikelets, then emasculate and bagg the panicle according to common methods;

B. pollination: pollinate the emasculated rice panicle with fresh maize pollens when the stigma of rice florets develops to mature;

C. production of haploid embryo: 24 h after the pollination, spray the maize-pollen-pollinated rice panicle with 2, 4-dichlorophenoxyacetic acid (2,4-D) solution, and continually keeping the rice panicle on maternal plants;

D. cutting of panicle and peeling of caryopsis: cutting the rice panicle off after the pollinated panicle has grown for 15-20 days, to collect caryopses formed through rice×maize hybridization; and

E. embryo rescue: dissect the sterilized caryopses on an aseptic bench by stereomicroscope to obtain haploid embryos, and inoculate the haploid embryos into the ½ MS medium, to obtain rice haploid plants after germination of the haploid embryos.

More preferably, in step C 24 h after the pollinating, the maize-pollen-pollinated rice panicle is sprayed with 2,4-D solution at concentration of 50-200 mg/L.

More preferably, the concentration of the 2,4-D solution is 100 mg/L.

More preferably, in step C the maize-pollen-pollinated rice plant is grown in an artificial climate room or an artificial climate chamber under artificial light (about 2 000 lux) 14 hours 27±1° C. and 10 hours 20±1° C. alternately, and keeping 85% humidity.

More preferably, in step D the pollinated panicle is cut off from the maternal plant when the length of the haploid embryos reach 0.5-1 mm.

The method for estimating the length of haploid embryos is: randomly dissecting several caryopsises before cutting off the panicle, where the caryopses are peeled under a dissecting microscope to find haploid embryos, and it is the optimum time point for cutting off the panicle when the embryos size are 0.5-1 mm.

More preferably, in the present invention, in step E, the culture medium is ½MS medium, and the haploid embryo is first placed in a culture chamber for dark culture (23±1° C.), and then subjected to light culture (about 2 000 lux,23±1° C.) after the haploid embryos germinate into buds, so as to obtain rice haploid plants.

It should be noted that rice haploid plants cannot bear seeds normally, and after the rice haploid plant is treated with chromosome doubling chemicals such as colchicine, the doubled haploid plants will be able to bear seeds normally.

The beneficial effects of the present invention: the method for producing rice haploids as provided by the present invention reduces the dependence on rice genotypes, does not produce mixture of haploid and diploid plants, contains no albino seedlings, and is simpler in technical operation.

### BRIEF DESCRIPTION OF THE DRAWINGS

To describe the technical solutions in the embodiments of the present invention or in the prior art more clearly, the following briefly describes the accompanying drawings required for describing the embodiments or the prior art. Apparently, the accompanying drawings in the following description show some embodiments of the present invention, and a person of ordinary skill in the art may still derive other drawings from these accompanying drawings without creative efforts.

FIG. 1 is a photograph showing the difference of caryopses between a maize pollen pollinated panicle (left) and a self-crossing panicle (right) in Example 1;

FIG. 2 is a comparative photograph of a normal diploid rice plant of Example 2 (left) and a haploid plant obtained in Example 2 (right);

FIG. 3 is a photograph showing a normal diploid rice plant of Example 3 (right) and a haploid plant obtained in Example 2 (left) in an artificial climate cabinet;

FIG. 4 is a photograph of the self-crossed caryopsis and the out-crossed caryopsis obtained through cultivation in Example 3, where in this figure, the left picture (having no embryo and endosperm) and the middle picture (having embryo but having no endosperm) are the out-crossed caryopsis, the right picture is the self-crossed caryopsis (having embryo and endosperm);

FIG. 5 is a photograph of an out-crossed caryopsis obtained in Example 3 and a haploid embryo thereof;

FIG. 6 is a diagram showing the results of chromosome ploidy detection on a normal diploid plant; and

FIG. 7 is a diagram showing the results of chromosome ploidy detection on a haploid plant obtained in Example 3.

FIG. 8 is a photograph showing differences in plant size and seed-set just before harvest between a normal rice plant (left) and a haploid rice plant (right) obtained in Example 3.

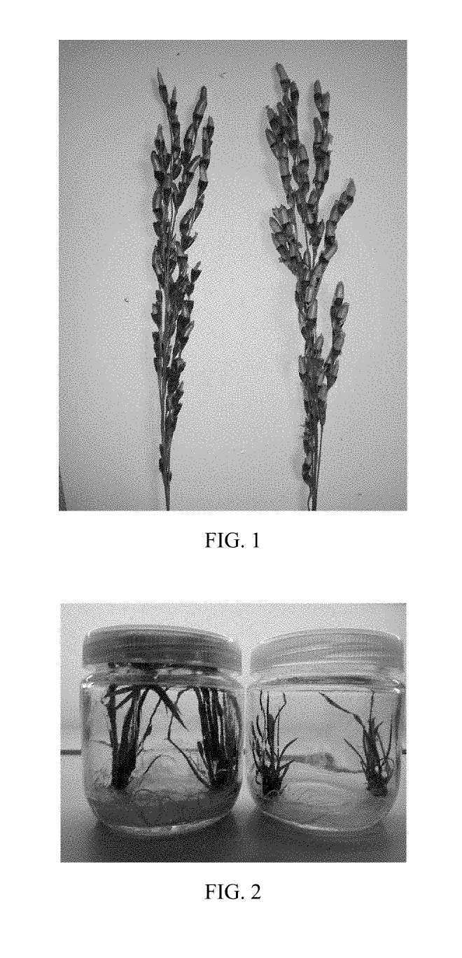

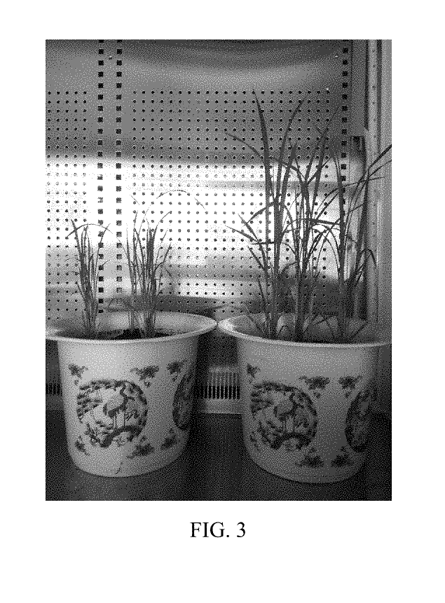

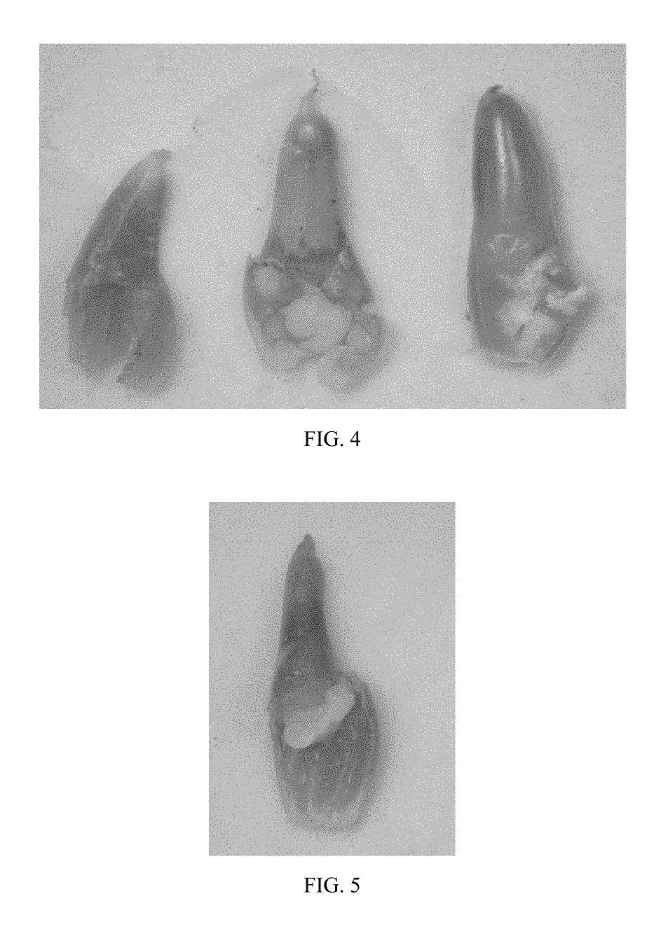

### DESCRIPTION OF THE EMBODIMENTS

In order to make the objectives, technical solutions and advantages of the present invention more apparent, the technical solution of present invention will be described in detail below. Apparently, the described embodiments are merely a part rather than all of the embodiments of the present invention. All other embodiments obtained by a person of ordinary skill in the art based on the embodiments of the present invention without creative efforts shall fall within the protection scope of the present invention.

In the present invention, all parts and percentages are units of weight, and all equipment and raw materials are commercially available or commonly used in the industry, unless otherwise specified. The methods in the following Examples are all routine methods in the art, unless otherwise specified.

### Example 1

A. material planting: a japonica rice variety Yunjing 37 (bred by Institute of Food Crops, Yunnan Academy of Agricultural Sciences, Yunnan, China) was subjected to sowed and seedlings raising in March, 2017, and transplanted into a pot in early May; and a maize (a maize inbred line SW6, (developed by Institute of Food Crops, Yunnan Academy of Agricultural Sciences, Yunnan, China) was sowed every 10 days from May to June, so that the flowering periods of the rice and the maize would meet from July to September, and thus hybridization pollination could be conducted.

B. emasculation: after heading and before flowering of the rice, for each panicle, cut off the young base spikelets and keep the upper and middle spikelets, and then the panicle was emasculated and bagged according to routine methods;

C. pollination: pollinating was conducted with fresh maize pollens when the stigma of rice florets developed to mature;

D. production of haploid embryos: 24 h after pollination, the maize-pollen-pollinated rice panicles were sprayed with 2,4-D solution at 50 mg/L, and the pollinated rice panicles were continually grown on their maternal plants;

E. cutting of panicle and peeling of caryopses: the rice panicle was cut off after the pollinated rice panicle had grown for 20 days, to collect caryopses produced from rice×maize hybridization; and

F. embryo rescue: the sterilized caryopses were peeled on an aseptic bench by stereomicroscope to obtain haploid embryos, and the haploid embryos were inoculated into the ½ MS medium, in which the haploid embryos were first darkly cultured, and then were subjected to light culture after the haploid embryos germinated into buds, to obtain green haploid plantlets.

### Example 2

A. material planting: an indica rice variety Liangyou 2186 (developed by Institute of Food Crops, Yunnan Academy of Agricultural Sciences, Yunnan, China) was sowed and seedlings raising in March, 2017, and transplanted into pots in early May; and a maize inbred line SW6 (developed by Institute of Food Crops, Yunnan Academy of Agricultural Sciences, Yunnan, China) was sowed every 10 days from May to June, so that the flowering periods of the rice and the maize would meet from July to September, and thus hybridization pollination could be conducted.

B. emasculation: after heading and before flowering of the rice, for each panicle, cut off the young base spikelets and keep the upper and middle spikelets, and then the panicle was emasculated and bagged according to routine methods;

C. pollination: pollinating was conducted with fresh maize pollens when the rice stigmas developed to mature;

D. production of haploid embryos: 24 h after pollination, the maize-pollen-pollinated rice panicles were sprayed with 2,4-D solution at 200 mg/L, and the pollinated rice panicles were continually grown on their maternal plants;

E. cutting of panicle and peeling of caryopses: the rice panicle was cut off after the pollinated rice panicle had grown for 18 days, to collect caryopses produced through rice×maize hybridization; and

F. embryo rescue: the sterilized caryopses were peeled on an aseptic bench by stereomicroscope to obtain haploid embryos, and the haploid embryos were inoculated into the ½ MS medium, in which the haploid embryos were first darkly cultured, and then were moved to light culture after the haploid embryos germinated into buds, to obtain green haploid plantlets.

### Example 3

A. material planting: a japonica rice variety Yunjing 37 (developed by Institute of Food Crops, Yunnan Academy of Agricultural Sciences, Yunnan, China) was sowed and seedlings raising in March, 2016, and transplanted into a pot in early May; and a maize inbred line SW6, developed by Institute of Food Crops, Yunnan Academy of Agricultural Sciences, Yunnan, China, was sowed every 10 days from May to June, so that the flowering periods of the rice and the maize would meet from July to September, and thus hybridization pollination could be conducted.

B. emasculation: after heading and before flowering of the rice, for each panicle, cut off the young base spikelets and keep the upper and middle spikelets, and then the panicle was emasculated and bagged according to routine methods;

C. pollination: pollinating was conducted with fresh maize pollens when the rice stigmas developed to mature;

D. production of haploid embryo: 24 h after pollination, the maize-pollen-pollinated rice panicles were sprayed with 2,4-D solution at 200 mg/L, and the pollinated rice panicles were continually grown on their maternal plants;

E. cutting of panicle and peeling of caryopses: the rice panicle was cut off after the pollinated rice panicle had grown for 15 days, to collect caryopses produced through rice×maize hybridization; and

F. embryo rescue: the sterilized caryopses were peeled on an aseptic bench by stereomicroscope to obtain haploid embryos, and the haploid embryos were inoculated into the ½ MS medium, in which the haploid embryos were first darkly cultured, and then were moved to light culture after the haploid embryos germinated into buds, to obtain green haploid plantlets.

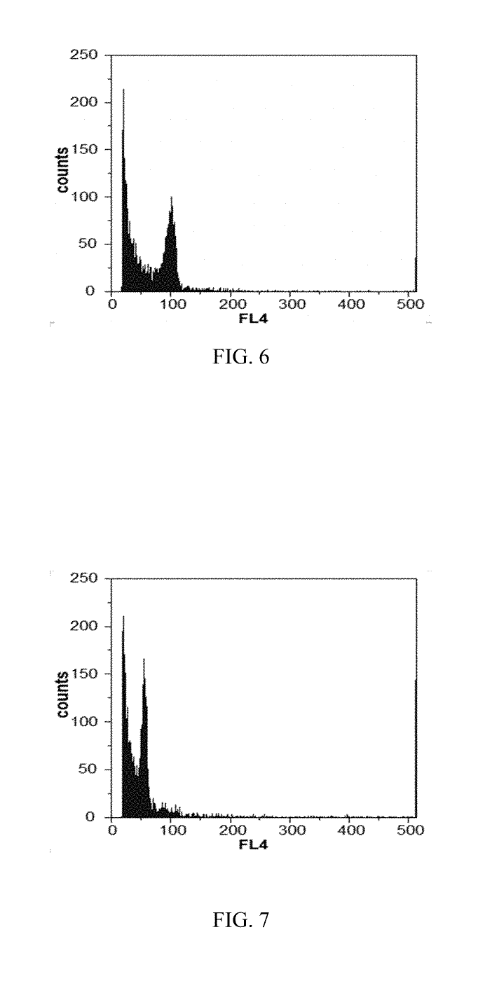

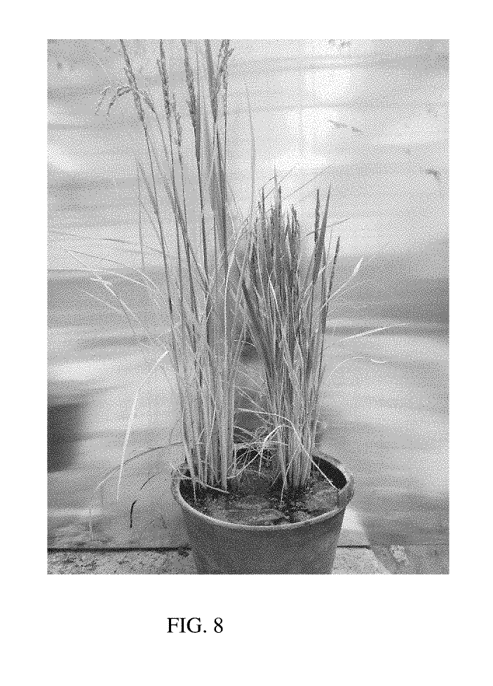

To demonstrate the rice plants obtained by the method of the present invention was haploid plants, the genome ploidy of the rice plants were detected by flow cytometry. The detection method was: first a cell nucleus was isolated, and then AT bases on the chromosomes were stained with DAPI dyeing solution, and then the intensity of fluorescence emitted by the stained AT bases were detected with a flow cytometer. The used instrument was CyFlow Space under the brand of Sysmex Partec, the kit was CyStain UV Precise P kit available from Sysmex Partec, and the ploidy of the samples could be determined based on the position of the DNA peak value detected by the flow cytometer. The DNA detection results of the normal diploid rice plant was shown in FIG. 6, and the DNA detection results of the rice plant obtained by the method of the present invention was shown in FIG. 7; it could be seen from FIG. 6 and FIG. 7 that, the ordinate (the number of isolated cell nucleuses) showed that the number of isolated cell nucleuses of the normal diploid rice plant to be detected was smaller than that of the plant produced by the present invention, and the abscissa (the DNA content) showed relatively higher main peaks in both the channel 100 and the channel 50 (the peak on the left side was an interference peak), and the abscissas in FIGS. 6 and 7 showed that the DNA content of the normal diploid rice plant was twice the DNA content of the rice plant produced by the method of the present invention, which proved that the rice plant produced by the method of the present invention was a rice haploid. In FIG. 8, the size of haploid rice plant obtained from the present invention was obviously smaller than that of normal diploid rice plant.

The aforementioned description is only specific embodiments of the present invention, and the claimed scope of the present invention is not limited thereto. Changes or substitutions can come into the mind of those of skills in the art readily, without departing from the technical scope disclosed by the present invention. These changes or substitutions all should fall within the claimed scope of the present invention. Therefore, the claimed scope of the present invention shall be determined by the claimed scope of the appended claims.

What is claimed is:

1. A method for producing the rice haploid by rice×maize hybridization, wherein the rice haploid is produced by the elimination of maize genome in wide cross between rice and maize.

2. The method for producing the rice haploid by rice×maize hybridization of claim 1, comprising the following steps:
A. emasculation: after heading and before flowering of the rice, for each panicle, cut off the young base spikelets and keep the upper and middle spikelets, and then the panicle is emasculated and bagged after emasculation according to routine methods;
B. pollination: the emasculated rice panicle is pollinated with fresh maize pollens when the stigmas of rice florets develop to mature;
C. production of haploid embryo: 24 h after the pollination, the pollinated rice panicle is sprayed with 2, 4-dichlorophenoxyacetic acid (2,4-D) solution, and continually grows on the maternal plant;
D. cutting of panicle and peeling of caryopsis: the pollinated rice panicle is cut off after growing on the maternal plant for 15-20 days, to collect the caryopses produced through rice×maize hybridization; and
E. embryo rescue: dissect the sterilized caryopses on the aseptic bench by stereomicroscope to obtain haploid embryos, and inoculate the haploid embryo into the ½ MS medium, to obtain the rice haploid plant after germination of the haploid embryo.

3. The method for producing the rice haploid by rice×maize hybridization of claim 2, wherein in step C, 24 h after the pollinating, the pollinated rice panicle is sprayed with 2,4-D solution at concentration of 50-200 mg/L.

4. The method for producing the rice haploid by rice×maize hybridization of claim 3, wherein in step C the pollinated rice plant is grown in an artificial climate room or an artificial climate chamber under artificial light (about 2 000 lux) 14 hours 27±1° C. or 10 hours 20±1° C. alternately, and keeping 85% humidity.

5. The method for producing the rice haploid by rice×maize hybridization of claim 2, wherein in step D, the pollinated panicle is cut off from the maternal plant when the length of the haploid embryos reach 0.5-1 mm.

6. The method for producing the rice haploid by rice×maize hybridization of claim 2, wherein in step E, the culture medium is ½MS medium, and the haploid embryos are first cultured under dark condition (23±1° C.), then moved to light condition (about 2 000 lux, 23±1° C.) after the haploid embryos germinate into buds, so as to obtain the rice haploid plant.
